(19) United States
(12) Patent Application Publication
(10) Pub. No.: US 2023/0255566 A1
(43) Pub. Date: Aug. 17, 2023

(54) DEVICE AND METHOD FOR ESTIMATING PHYSIOLOGICAL PARAMETER VALUE, MONITORING DEVICE, AND NON-TRANSITORY COMPUTER-READABLE MEDIUM

(71) Applicant: NIHON KOHDEN CORPORATION, Tokyo (JP)

(72) Inventors: Yoshihiro SUGO, Tokorozawa-shi, Saitama (JP); Yoshiharu HARADA, Tokorozawa-shi, Saitama (JP); Mami TERAO, Tokorozawa-shi, Saitama (JP); Sayaka YAMAZAKI, Tokorozawa-shi, Saitama (JP)

(73) Assignee: NIHON KOHDEN CORPORATION, Tokyo (JP)

(21) Appl. No.: 18/015,281

(22) PCT Filed: Jul. 14, 2021
(86) PCT No.: PCT/JP2021/026444

§ 371 Date: Jan. 9, 2023

(30) Foreign Application Priority Data
Jul. 15, 2020 (JP) 2020-121471

(51) Int. Cl. A61B 5/00; A61B 5/0205; G16H 50/30

(52) U.S. Cl. CPC A61B 5/7278; A61B 5/0205; G16H 50/30; A61B 5/021

(57) ABSTRACT

A first input interface receives measured values of non-invasive blood pressure of a subject intermittently at a first frequency. A second input interface receives measured values of pulse wave transit time of the subject at a second frequency higher than the first frequency. A processor executes regression analysis processing based on the measured values received until when a measured value acquired by Nth (N is an integer that is no less than 2) measurement of the non-invasive blood pressure is received to acquire a regression equation in which the measured value is set as an explanatory variable. An information presenting device visibly presents estimated values of the non-invasive blood pressure calculated with the regression equation and the measured values, until a measured value acquired by (N+1)th measurement of the non-invasive blood pressure is received.

### TECHNICAL FIELD

The presently disclosed subject relates to an estimating device and method for estimating a physiological parameter value of a subject. The presently disclosed subject matter also relates to a monitoring device equipped with the estimating device, as well as a non-transitory computer-readable medium having stored a computer program adapted to be executed by a processor in the estimating device.

### BACKGROUND

Japanese Patent Publication No. 2009-508577A discloses an apparatus for non-invasively measuring a blood pressure value that is an example of the physiological parameter value of the subject. Since the measurement is performed while changing an internal pressure of a cuff attached to an arm or the like of the subject, it is inevitable that measured values are acquired intermittently.

### SUMMARY

### Technical Problem

It is demanded to provide a technique that spuriously realizes constant monitoring of a physiological parameter value while measuring the physiological parameter value in an intermittent manner.

### Solution to Problem

In order to meet the above demand, a first illustrative aspect of the presently disclosed subject matter provides an estimating device for a physiological parameter value, comprising:
a first interface configured to receive measured values of a first physiological parameter of a subject intermittently at a first frequency;
a second interface configured to receive measured values of n (n is an integer that is no less than 1) second physiological parameter of the subject at a second frequency higher than the first frequency; and
a processor configured to:
   execute regression analysis processing based on the measured values of the second physiological parameter received by the second interface until when a measured value acquired by Nth (N is an integer that is no less than n+1) measurement of the first physiological parameter is received by the first interface in order to acquire a regression equation in which the measured value of the second physiological parameter is set as an explanatory variable;
   calculate estimated values of the first physiological parameter with the regression equation and the measured values of the second physiological parameter received by the second interface, until a measured value acquired by (N+1)th measurement of the first physiological parameter is received by the first interface; and
   output data for visibly presenting the estimated values.

In order to meet the above demand, a second illustrative aspect of the presently disclosed subject matter provides a non-transitory computer-readable medium having stored a computer program adapted to be executed by a processor in an estimating device configured to estimate a physiological parameter value of a subject, the computer program being configured to cause, when executed, the estimating device to:
receive measured values of a first physiological parameter of a subject intermittently at a first frequency;
receive measured values of n (n is an integer that is no less than 1) second physiological parameter of the subject at a second frequency higher than the first frequency;
execute regression analysis processing based on the measured values of the second physiological parameter received until when a measured value acquired by Nth (N is an integer that is no less than n+1) measurement of the first physiological parameter is received in order to acquire a regression equation in which the measured value of the second physiological parameter is set as an explanatory variable;
calculate estimated values of the first physiological parameter with the regression equation and the measured values of the second physiological parameter, until a measured value acquired by (N+1)th measurement of the first physiological parameter is received by the first interface; and
output data for visibly presenting the estimated values.

In order to meet the above demand, a third illustrative aspect of the presently disclosed subject matter provides a monitoring device, comprising:
a first interface configured to receive measured values of a first physiological parameter of a subject intermittently at a first frequency;
a second interface configured to receive measured values of n (n is an integer that is no less than 1) second physiological parameter of the subject at a second frequency higher than the first frequency;
a processor configured to execute regression analysis processing based on the measured values of the second physiological parameter received by the second interface until when a measured value acquired by Nth (N is an integer that is no less than n+1) measurement of the first physiological parameter is received by the first interface in order to acquire a regression equation in which the measured value of the second physiological parameter is set as an explanatory variable; and
an information presenting device configured to visibly present estimated values of the first physiological parameter calculated with the regression equation and the measured values of the second physiological parameter received by the second interface, until a measured value acquired by (N+1)th measurement of the first physiological parameter is received by the first interface.

In order to meet the above demand, a fourth illustrative aspect of the presently disclosed subject matter provides an estimating method for a physiological parameter value, comprising:
receiving measured values of a first physiological parameter of a subject intermittently at a first frequency;
receiving measured values of n (n is an integer that is no less than 1) second physiological parameter of the subject at a second frequency higher than the first frequency; executing regression analysis processing based on the measured values of the second physiological parameter received until when a measured value acquired by Nth (N is an integer that is no less than n+1) measurement of the first physiological parameter is received in order to acquire a regression equation in which the measured value of the second physiological parameter is set as an explanatory variable; and
presenting visibly estimated values of the first physiological parameter calculated with the regression equation and the measured values of the second physiological parameter, until a measured value acquired by (N+1)th measurement of the first physiological parameter is received by the first interface.

According to the configuration of each of the illustrative aspects, in a case where the number of the second physiological parameter is “n”, estimated values of the first physiological parameter can be calculated with a regression equation acquired by the regression analysis processing after (n+1)th measurement of the first physiological parameter is performed. The calculation frequency of the estimated values can be made higher than the acquisition frequency of the measured values of the first physiological parameter. Accordingly, by visibly presenting the estimated values of the first physiological parameter so as to complement measurements of the first physiological parameter that shall be performed intermittently, it is possible to cause a user to experience a situation as if the first physiological parameter is measured constantly. In other words, it is possible to realize constant monitoring of the first physiological parameter spuriously, even if the first physiological parameter is measured in an intermittent manner.

### BRIEF DESCRIPTION OF DRAWINGS

FIG. 1 illustrates a functional configuration of a monitoring device according to an embodiment.

FIG. 2 illustrates a flow of processing executed by a processor of FIG. 1.

FIG. 3 illustrates an example of regression analysis processing executed by the processor of FIG. 1.

FIG. 4 illustrates visible presentation performed by an information presenting device of FIG. 1 on the basis of the regression analysis processing of FIG. 3.

FIG. 5 illustrates another example of regression analysis processing executed by the processor of FIG. 1.

FIG. 6 illustrates visible presentation performed by the information presenting device of FIG. 1 on the basis of the regression analysis processing of FIG. 5.

FIG. 7 illustrates another example of regression analysis processing executed by the processor of FIG. 1.

FIG. 8 illustrates another example of regression analysis processing executed by the processor of FIG. 1.

FIG. 9 illustrates visible presentation performed by the information presenting device of FIG. 1 on the basis of the regression analysis processing of FIG. 8.

FIG. 10 illustrates another example of regression analysis processing executed by the processor of FIG. 1.

FIG. 11 illustrates an example of relationship between an optimum number of samples and the number of measurements that is visibly presented by the information presenting device of FIG. 1.

FIG. 12 illustrates another example of relationship between an optimum number of samples and the number of measurements that is visibly presented by the information presenting device of FIG. 1.

FIG. 13 illustrates statistics that are visibly presented by the information presenting device of FIG. 1.

FIG. 14 illustrates another example of relationship between the monitoring device and an estimating device.

### DESCRIPTION OF EMBODIMENTS

Examples of embodiments will be described in detail below with reference to the accompanying drawings. In the drawings, the scale is appropriately changed in order to make each element to be described have a recognizable size.

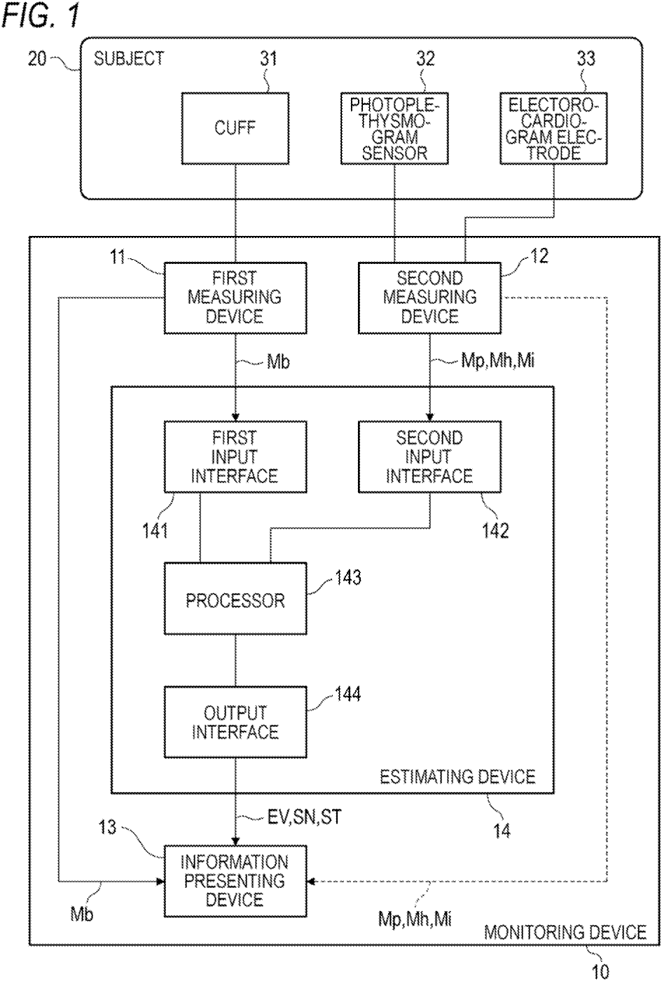

FIG. 1 illustrates a functional configuration of a monitoring device 10 according to an embodiment. The monitoring device 10 includes a first measuring device 11, a second measuring device 12, an information presenting device 13, and an estimating device 14.

The first measuring device 11 is configured to acquire a measured value Mb of a non-invasive blood pressure of the subject 20 with a cuff 31 attached to the body of the subject 20. The non-invasive blood pressure is an example of a first physiological parameter. Since a configuration itself in which the non-invasive blood pressure is measured by using the cuff 31 is well-known, detailed descriptions thereof will be omitted.

The second measuring device 12 is configured to acquire a measured value Mp of a pulse wave transit time, a measured value Mh of a heart rate, and a measured value Mi of a perfusion index (including a logarithmic value thereof) with a photoplethysmogram sensor 32 and an electrocardiogram electrode 33 that are attached to a body of the subject 20. Each of the pulse wave transit time, the heart rate, and the perfusion index is an example of a second physiological parameter. The pulse wave transit time is a time period from a time point corresponding to a peak of an R wave in the electrocardiogram to a time point at which a pulse wave appears in an extremity. The perfusion index is calculated based on a waveform magnitude of the pulse wave as a numerical value of a ratio of a pulsatile component and a non-pulsatile component in the waveform. At least when the pulse wave transit time is measured, the photoplethysmogram sensor 32 and the electrocardiogram electrode 33 are both used. Since a configuration itself in which each of the pulse wave transit time, the heart rate, and the perfusion index is measured is well known, detailed descriptions thereof will be omitted.

For example, the non-invasive blood pressure may be measured by the first measuring device 11 every 5 minutes. The pulse wave transit time, the heart rate, and the perfusion index may be measured by the second measuring device 12 every one minute. In this case, the non-invasive blood pressure, the pulse wave transit time, the heart rate, and the perfusion index are measured at the same time every 5 minutes. In other words, the measured values of the pulse wave transit time, the heart rate, and the perfusion index are acquired at a higher frequency than the measured value of the non-invasive blood pressure. Once every 5 minutes is an example of a first frequency. Once a minute is an example of a second frequency.

The information presenting device 13 is configured to visibly present at least one of the measured value Mb of the non-invasive blood pressure and changes over time thereof. The changes over time of the measured value may be presented in the form of a graph or a chart. The visible presentation may be performed with a display or a screen, or may be performed with printing on a paper medium. The information presenting device 13 may visibly present at least one of the measured value and the changes over time thereof with respect to each of the pulse wave transit time, the heart rate, and the perfusion index.

The estimating device 14 is configured to calculate an estimated value Eb of the non-invasive blood pressure based on the measured value Mp of the pulse wave transit time, the measured value Mh of the heart rate, and the measured value Mi of the perfusion index. The estimating device 14 includes a first input interface 141, a second input interface 142, a processor 143, and an output interface 144.

The first input interface 141 is configured as an interface that receives the measured value Mb of the non-invasive blood pressure from the first measuring device 11. The measured value Mb may be in the form of analog data or digital data. In the case where the measured value Mb is in the form of analog data, the first input interface 141 is configured to be equipped with an adequate conversion circuit including an A/D converter.

The second input interface 142 is configured as an interface that receives, from the second measuring device 12, the measured value Mp of the pulse wave transit time, the measured value Mh of the heart rate, and the measured value Mi of the perfusion index. Each of the measured value Mp, the measured value Mh, and the measured value Mi may be in the form of analog data or digital data. In the case where each of the measured value Mp, the measured value Mh, and the measured value Mi is in the form of analog data, the second input interface 142 is configured to be equipped with an adequate conversion circuit including an A/D converter.

A correlation may be established between the non-invasive blood pressure, and each of the pulse wave transit time, the heart rate, and the perfusion index. The processor 143 is configured to execute a regression analysis processing wherein each of the pulse wave transit time, the heart rate, and the perfusion index is set as an explanatory variable, and the non-invasive blood pressure is set as an objective variable. In addition, the processor 143 is configured to calculate an estimated value Eb of the non-invasive blood pressure based on at least one regression equation that is acquired by the regression analysis processing, and the measured values of the pulse wave transit time, the heart rate, and the perfusion index.

The output interface 144 is configured as an interface that outputs estimated value data EV for causing the information presenting device 13 to visibly present the estimated value Eb of the non-invasive blood pressure that is calculated by the processor 143. The estimated value data EV may be in the form of analog data or digital data. In the case where the estimated value data EV is in the form of analog data, the output interface 144 is configured to be equipped with an adequate conversion circuit including a D/A converter.

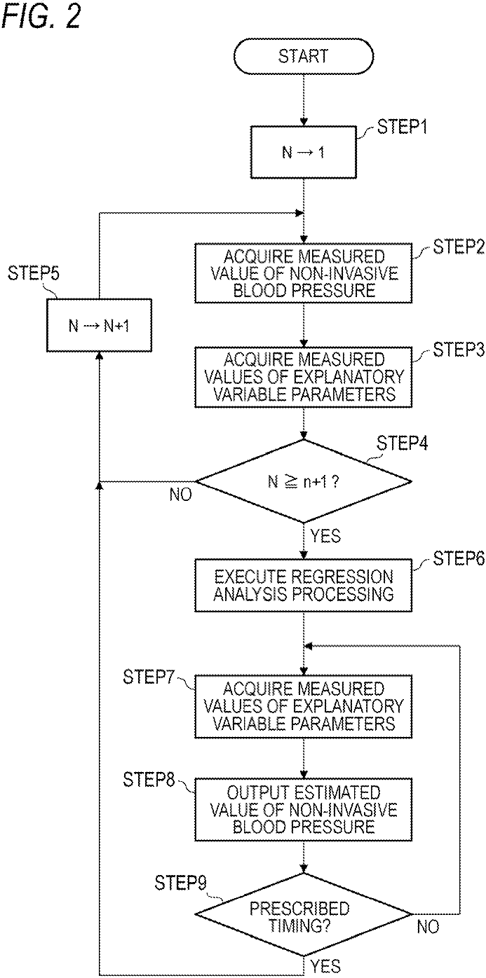

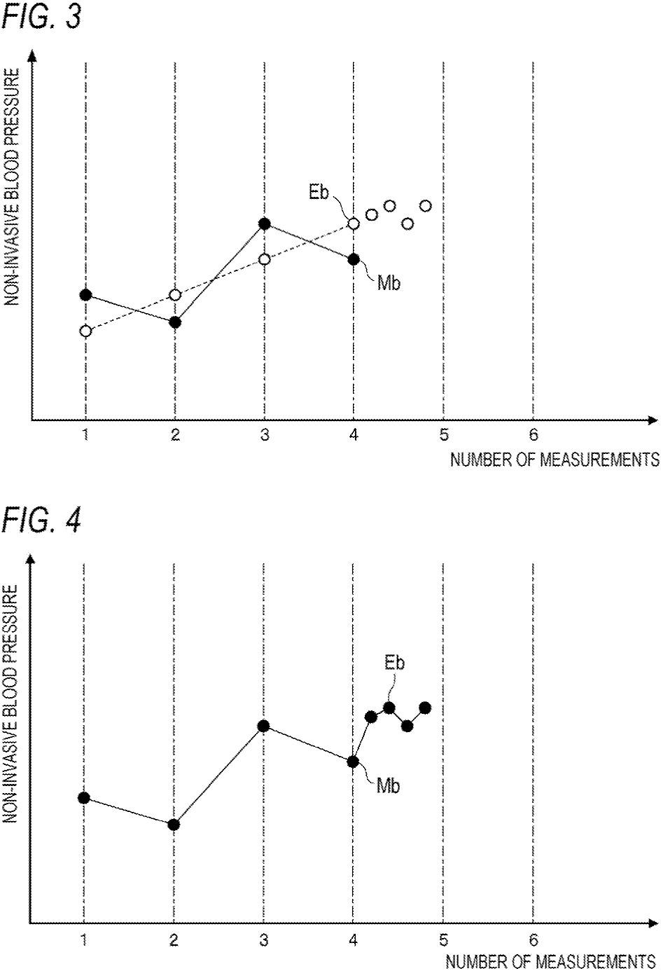

Next, the processing performed by the processor 143 will be described in detail with reference to FIGS. 2 and 3.

The processor 143 includes an internal counter that counts the number of measurements N of the non-invasive blood pressure performed by the first measuring device 11. First, in STEP1, the processor 143 sets 1 to a value of the number of measurements N.

When the first measuring device 11 performs measurement of the first non-invasive blood pressure, the processor 143 acquires the measured value Mb of the non-invasive blood pressure through the first input interface 141 (STEP2). As described above, the second measuring device 12 performs measurement of the pulse wave transit time, the heart rate, and the perfusion index at the same time. The processor 143 acquires, through the second input interface 142, the measured value Mp of the pulse wave transit time, the measured value Mh of the heart rate, and the measured value Mi of the perfusion index (STEP3).

Subsequently, the processor 143 determines whether the value of the number of measurements N indicated by the internal counter is no less than (n+1) (STEP4). The “n” is the number of different the explanatory variable parameters used in the regression analysis processing. In this example, since the number of kinds is 3, the processor 143 determines whether the value of the number of measurements N is no less than 4. This is because, in order to execute the regression analysis processing with 3 different explanatory variable parameters, at least 4 sets of measured values consisting of the measured value Mb of the non-invasive blood pressure, the measured value Mp of the pulse wave transit time, the measured value Mh of the heart rate, and the measured value Mi of the perfusion index are necessary.

In a case where the value of the number of measurements N is less than 4 (NO in STEP4), the processor 143 adds 1 to the value of the internal counter (STEPS), and returns the processing to STEP2. The processing of STEP2 to STEPS is repeated until 4th non-invasive blood pressure measurement is performed.

In a case where the value of the number of measurements N is no less than 4 (YES in STEP4), the processor 143 executes the regression analysis processing (STEP6). In the present example wherein 3 different explanatory variable parameters are used, the following 7 regression equations may be acquired in order to calculate the estimated value Eb of the non-invasive blood pressure.

$$\text{Eb} \text{=} \text{k0} \text{+} \text{k1} ⋅ \text{Mp} \qquad \text{(1)}$$

$$\text{Eb} \text{=} \text{k0} \text{+} \text{k2} ⋅ \text{Mh} \qquad \text{(2)}$$

$$\text{Eb} \text{=} \text{k0} \text{+} \text{k3} ⋅ \text{Mi} \qquad \text{(3)}$$

$$\text{Eb} \text{=} \text{k0 + k1} ⋅ \text{Mp + k2} ⋅ \text{Mh} \qquad \text{(4)}$$

$$\text{Eb} \text{=} \text{k0 + k1} ⋅ \text{Mp + k3} ⋅ \text{Mi} \qquad \text{(5)}$$

$$\text{Eb} \text{=} \text{k0 + k2} ⋅ \text{Mh + k3} ⋅ \text{Mi} \qquad \text{(6)}$$

$$\text{Eb} \text{=} \text{k0 + k1} ⋅ \text{Mp + k2} ⋅ \text{Mh} \text{+} \text{k3} ⋅ \text{Mi} \qquad \text{(7)}$$

Here, k0 is a constant term, and each of k1, k2, and k3 is a partial regression coefficient.

Until the next measurement of the non-invasive blood pressure is performed, the processor 143 acquires, through the second input interface 142, the measured value Mp of the pulse wave transit time, the measured value Mh of the heart rate, and the measured value Mi of the perfusion index (STEP7).

The processor 143 substitutes at least one of the measured value Mp of the pulse wave transit time, the measured value Mh of the heart rate, and the measured value Mi of the perfusion index into at least one of the regression equations that are acquired by the regression analysis processing, thereby calculating the estimated value Eb of the non-invasive blood pressure. The processor 143 outputs, from the output interface 144, the estimated value data EV that causes the information presenting device 13 to visibly present the estimated value Eb as calculated (STEP8).

Subsequently, in STEP9, the processor 143 determines whether a prescribed timing has arrived. Specifically, it is determined whether a next measurement timing of the non-invasive blood pressure that is performed every 5 minutes has arrived. When it is determined that the prescribed timing has not arrived (NO in STEP9), the processor 143 returns the processing to STEP7. Accordingly, until the next measurement timing of the non-invasive blood pressure arrives, the measured value Mp of the pulse wave transit time, the measured value Mh of the heart rate, and the measured value Mi of the perfusion index are acquired, and the calculation of the estimated value Eb of the non-invasive blood pressure is repeated.

When it is determined that the prescribed timing has arrived (YES in STEP9), the processor 143 adds 1 to the value of the internal counter (STEP5), and returns the processing to STEP2. As a result, the regression analysis processing and the calculation of the estimated value Eb of the non-invasive blood pressure are repeated based on 5th and later measurements of the non-invasive blood pressure.

FIG. 3 illustrates a case where only one of the 7 regression equations is used. In this figure, each of a plurality of black circles connected by solid lines represents a measured value Mb of the non-invasive blood pressure. A dashed line represents a regression equation acquired by the regression analysis processing executed when the 4th non-invasive blood pressure measurement is performed. Each of a plurality of white circles represents an estimated value Eb of the non-invasive blood pressure that is calculated with the regression equation.

During a time period from when the 4th measurement of the non-invasive blood pressure is performed to when the 5th measurement of the non-invasive blood pressure is performed, the second measuring device 12 performs measurements of the pulse wave transit time, the heart rate, and the perfusion index every one minute (4 times in total). The processor 143 calculates an estimated value Eb of the non-invasive blood pressure with the regression equation that is acquired through the regression analysis processing every time the second measuring device 12 performs the measurement. Accordingly, in the example illustrated in FIG. 3, four estimated values Eb are calculated during the time period from when the 4th measurement of the non-invasive blood pressure is performed to when the 5th measurement of the non-invasive blood pressure is performed.

The time interval for which measurements of the pulse wave transit time, the heart rate, and the perfusion index are performed with the photoplethysmogram sensor 32 and the electrocardiogram electrode 33 may be further shortened. In this case, it is possible to increase the number of the estimated values Eb of the non-invasive blood pressure that can be calculated while two measurements of the non-invasive blood pressure are performed.

FIG. 4 illustrates a state where, based on the estimated value data EV outputted from the output interface 144 of the estimating device 14, the estimated value Eb of the non-invasive blood pressure calculated as described above is visibly presented together with the measured values Mb of the non-invasive blood pressure in the information presenting device 13 of the monitoring device 10.

According to the above configuration, in a case where the number of the explanatory variables is “n”, estimated values Eb of the non-invasive blood pressure can be calculated with a regression equation acquired by the regression analysis processing after (n+1)th measurement of the non-invasive blood pressure is performed. The calculation frequency of the estimated values Eb can be made higher than the acquisition frequency of the measured values Mb of the non-invasive blood pressure. Accordingly, by visibly presenting the estimated values Eb of the non-invasive blood pressure so as to complement measurements of the non-invasive blood pressure that shall be performed intermittently, it is possible to cause a user to experience a situation as if the non-invasive blood pressure is measured constantly. In other words, it is possible to realize constant monitoring of the non-invasive blood pressure spuriously, even if the non-invasive blood pressure is measured in an intermittent manner.

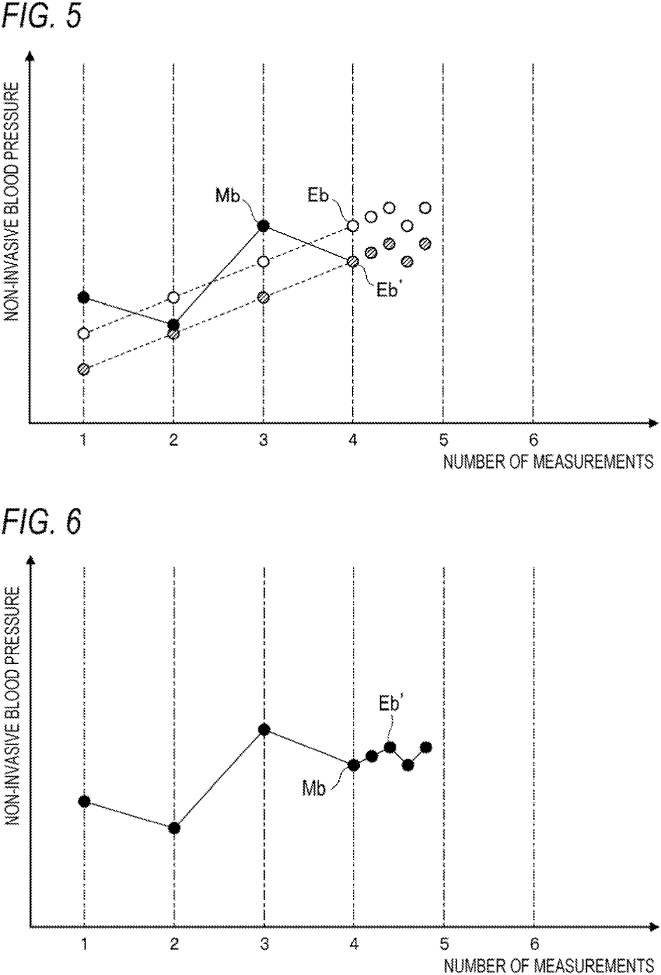

As illustrated in FIG. 5, the processor 143 of the estimating device 14 may be configured to perform constant term correction on the regression equation acquired by the regression analysis processing. Specifically, the processor 143 calculates an estimated value Eb of the non-invasive blood pressure at a time point when the 4th measurement of the non-invasive blood pressure is performed, with the regression equation acquired by the regression analysis processing. Subsequently, the processor 143 corrects the constant term k0 of the regression equation so that the estimated value Eb as calculated coincides with the measured value Mb acquired by the 4th measurement of the non-invasive blood pressure. Hatched circles illustrated in FIG. 5 represent estimated values Eb′ of the non-invasive blood pressure that is calculated with the regression equation for which the constant term correction has been performed.

FIG. 6 illustrates a state where the estimated values Eb′ of the non-invasive blood pressure calculated as described above are visibly presented in the information presenting device 13 of the monitoring device 10 together with the measured values Mb of the non-invasive blood pressure, based on the estimated value data EV outputted from the output interface 144 of the estimating device 14.

With the above configuration, correction is performed so as to suppress the difference between the estimated value Eb of the non-invasive blood pressure calculated with the regression equation acquired by the regression analysis processing and the latest measured value Mb of the non-invasive blood pressure. Accordingly, it is possible to enhance accuracy for the calculations of the estimated values Eb of the non-invasive blood pressure that are to be performed until the next measurement of the non-invasive blood pressure. As a result, as is apparent from a comparison between FIGS. 4 and 6, it is possible to enhance natural continuity between a portion where the measured values Mb are visibly presented and a portion where the estimated values Eb are visibly presented.

As described above, in this example, 7 regression equations are acquired by the regression analysis processing. Therefore, the processor 143 of the estimating device 14 can calculate 7 different estimated values Eb of the non-invasive blood pressure by using at most 7 different regression equations. In this case, the processor 143 is configured to perform processing for selecting one optimum regression equation in order to obtain an estimated value Eb that is to be subjected to the visible presentation in the information presenting device 13 of the monitoring device 10.

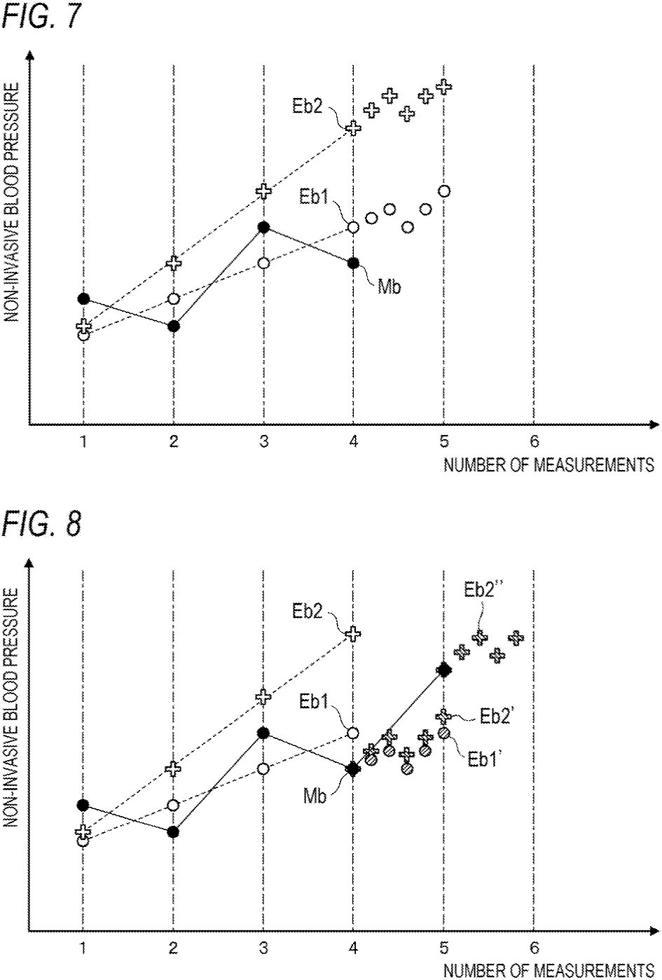

FIG. 7 illustrates a case where estimated value of the non-invasive blood pressure are calculated with two regression equations. Each of the white circles represents an estimated value Eb1 of the non-invasive blood pressure calculated with the first regression equation. The first regression equation is the same as the regression equation described with reference to FIGS. 3 to 6. Each of the crosses represents an estimated value Eb2 of the non-invasive blood pressure that is calculated with the second regression equation.

In this case, the processor 143 is configured to select a regression equation used to calculate an estimated value that is closer to the measured value Mb acquired by the 4th measurement of the non-invasive blood pressure. In the example illustrated in FIG. 7, the estimated value Eb1 is closer to the measured value Mb than the estimated value Eb2. Accordingly, the processor 143 selects the first regression equation and outputs, from the output interface 144, estimated value data EV in accordance with the estimated value Eb1. As a result, the estimated values Eb1 that are to be visibly presented in the information presenting device 13 of the monitoring device 10 can be made similar to those illustrated in FIG. 4. In a case where the constant term correction is performed on the first regression equation, the estimated values Eb1 that are to be visibly presented in the information presenting device 13 of the monitoring device 10 is made similar to those illustrated in FIG. 6.

As illustrated in FIG. 8, the selection of the regression equation used for calculating the estimated values of the non-invasive blood pressure that are to be subjected to the visible presentation may be performed when measurement of the non-invasive blood pressure is performed subsequently or later to the measurement of the non-invasive blood pressure by which the regression analysis processing is first enabled. As described above, at the time point when the 4th measurement of the non-invasive blood pressure is performed, the estimated value Eb1 of the non-invasive blood pressure calculated with the first regression equation is closer to the measured value Mb acquired by the 4th measurement of the non-invasive blood pressure than the estimated value Eb2 of the non-invasive blood pressure calculated with the second regression equation. In this example, no optimum regression equation is selected at this time point, and two different estimated values are calculated with the two regression equations until the 5th measurement of the non-invasive blood pressure measurement is performed.

In the example illustrated in FIG. 8, the constant term correction is performed on each regression equation, thereby illustrating an estimated value Eb1′ of the non-invasive blood pressure calculated with the first regression equation as corrected, and an estimated value Eb2′ of the non-invasive blood pressure calculated with the second regression equation. However, the following descriptions can be applied also to a case where the constant term correction is omitted.

When the 5th measurement of the non-invasive blood pressure is performed, the processor 143 of the estimating device 14 compares each of the estimated value Eb1′ and the estimated value Eb2′ calculated with the two regression equations acquired by the regression analysis processing executed when the 4th measurement of the non-invasive blood pressure is performed, with the measured value Mb acquired by the 5th measurement of the non-invasive blood pressure. Subsequently, the processor 143 selects a regression equation used to calculate an estimated value that is closer to the 5th measured value Mb of the non-invasive blood pressure.

In the example illustrated in FIG. 8, the estimated value Eb2′ of the non-invasive blood pressure calculated with the second regression equation is closer to the measured value Mb acquired by the 5th measurement of the non-invasive blood pressure than the estimated value Eb1′ of the non-invasive blood pressure calculated with the first regression equation. Accordingly, the processor 143 selects the second regression equation. Until the 6th measurement of the non-invasive blood pressure is performed, the processor calculates estimated values of the non-invasive blood pressure with the second regression equation, and outputs estimated value data EV in accordance with the estimated values from the output interface 144. In this example, since the constant term correction is performed the second regression equation as selected, the estimated value of the non-invasive blood pressure calculated with the second regression equation as corrected is assigned with a reference symbol Eb2″ to be distinguished from the estimated value Eb2′.

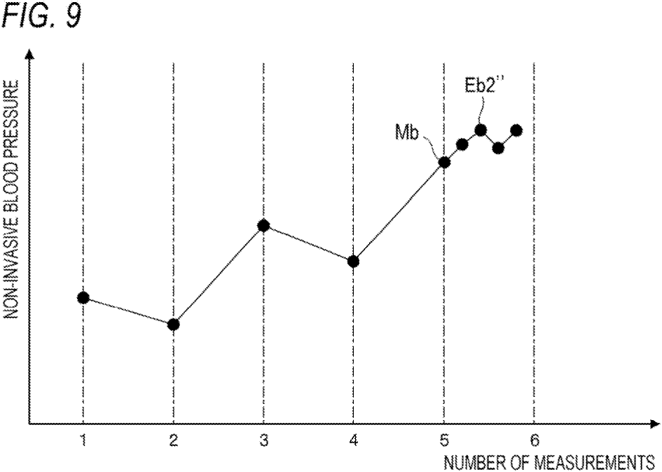

FIG. 9 illustrates a state where the estimated values Eb2″ of the non-invasive blood pressure calculated as described above are visibly presented in the information presenting device 13 of the monitoring device 10 together with the measured values Mb of the non-invasive blood pressure, based on the estimated value data EV outputted from the output interface 144 of the estimating device 14.

According to the above configuration, it is possible to evaluate the regression equation with reference to a change of the measured value Mb of the non-invasive blood pressure that has occurred since when the regression analysis processing is enabled to be executed. Accordingly, it is possible to more dynamically select an optimum regression equation for calculating the estimated value Eb of the non-invasive blood pressure.

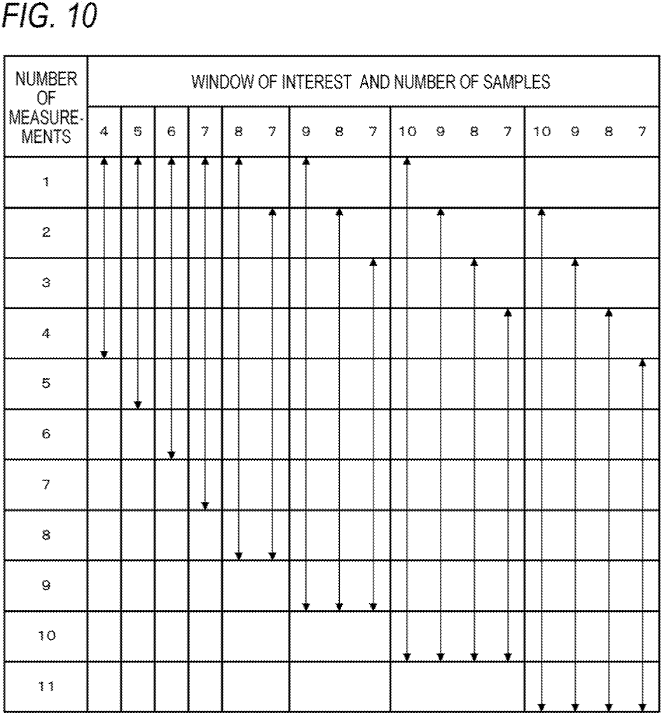

As illustrated in FIG. 10, the processor 143 of the estimating device 14 may change at least one of a window of interest designating which measurements are to be subjected to the regression analysis processing and number of measurements to be subjected to the regression analysis processing as samples.

As described above, in the case where 3 explanatory variable parameters are used, the number of samples required for performing the regression analysis processing is at least 4. Accordingly, in the regression analysis processing executed when the 5th measurement of the non-invasive blood pressure is performed, at least 4 measurements included in a window of interest designating the 2nd to 5th measurements may be sampled. In the example illustrated in FIG. 10, 5 measurements included in a window of interest designating the 1st to 5th measurements are sampled for the regression analysis processing executed when the 5th measurement of the non-invasive blood pressure is performed. Similarly, until the 7th measurement of the non-invasive blood pressure is performed, both the window of interest and the number of samples for the regression analysis processing are increased. However, for the regression analysis processing executed when the 7th measurement of the non-invasive blood pressure is performed, 4 measurements included in a window of interest designating the 4th to 7th measurements may be sampled.

By increasing the window of interest and the number of samples, it is possible to enhance the calculation accuracy of the estimated value of the non-invasive blood pressure based on the regression equation acquired by the regression analysis processing. In a case where the regression analysis processing is executed such that a later measurement is included in the window of interest while suppressing an increase in the number of samples, it is possible to cause a later state of the non-invasive blood pressure to be well reflected to the result of the processing.

The processor 143 of the estimating device 14 may be configured to perform the regression analysis processing plural times while changing the number of samples (the measured value Mb of the non-invasive blood pressure) to be used, thereby acquiring at least one regression equation every time the regression analysis processing is executed. In the example illustrated in FIG. 10, two different regression analysis processings are performed in the 8th measurement of the non-invasive blood pressure. In the first regression analysis processing, 8 measurements included in the window of interest designating the 1st to 8th measurements are sampled. In the second regression analysis processing, 7 measurements included in the window of interest designating the 2nd to 8th measurements are sampled.

The at least one regression equation acquired by the first regression analysis processing coincides with the at least one regression equation acquired by the second regression analysis processing. For example, in a case where the equations (6) and (7) among the 7 regression equations available in this example are acquired by the first regression analysis, the equations (6) and (7) are acquired by the second regression analysis processing as well.

In this example, similarly to the example described with reference to FIG. 7, the processor 143 is configured to select one regression equation used to calculate an estimated value that is closer to the measured value Mb acquired by the 8th measurement of the non-invasive blood pressure from the regression equations acquired by two different regression analysis processings. In this case, the processor 143 is configured to output, from the output interface 144, estimated value data EV for visibly presenting, in the information presenting device 13 of the monitoring device, estimated values Eb of the non-invasive blood pressure calculated with the regression equation as selected, until the 9th measurement of the non-invasive blood pressure is performed.

Alternatively, similarly to the example described with reference to FIG. 8, the processor 143 is configured to select one regression equation used to calculate an estimated value that is closer to the measured value Mb acquired by the 9th measurement of the non-invasive blood pressure from the regression equations acquired by two different regression analysis processings. In this case, the processor 143 is configured to output, from the output interface 144, estimated value data EV for visibly presenting, in the information presenting device 13 of the monitoring device, estimated values Eb of the non-invasive blood pressure calculated with the regression equation as selected, until the 10th measurement of the non-invasive blood pressure is performed.

According to the above configuration, not only a regression equation that enables calculation of the estimated value Eb of the non-invasive blood pressure with higher accuracy can be selected, but also information as for the optimum window of interest and the optimum number of samples related to the regression analysis processing can be acquired.

The processor 143 of the estimating device 14 may be configured to increase the number of regression analysis processings to be performed in accordance with an increase in the number of measurements N.

In the example illustrated in FIGS. 10, 3 different regression analysis processings are performed when the 9th measurement of the non-invasive blood pressure. In the first regression analysis processing, 9 measurements included in the window of interest designating the 1st to 9th measurements are sampled. In the second regression analysis processing, 8 measurements included in the window of interest designating the 2nd to 9th measurements are sampled. In the third regression analysis processing, 7 measurements included in the window of interest designating the 3rd to 9th measurements are sampled.

4 different regression analysis processings are performed when the 10th measurement of the non-invasive blood pressure. In the first regression analysis processing, 10 measurements included in the window of interest designating the 1st to 10th measurements are sampled. In the second regression analysis processing, 9 measurements included in the window of interest designating the 2nd to 10th measurements are sampled. In the third regression analysis processing, 8 measurements included in the window of interest designating the 3rd to 10th measurements are sampled. In the fourth regression analysis processing, 7 measurements included in the window of interest designating the 4th to 10th measurements are sampled.

How to select one optimum regression equation from the regression equations acquired by the regression analysis processings is similar to the example mentioned with reference to the 8th measurement of the non-invasive blood pressure.

As the number of measurements N of the non-invasive blood pressure is increased, options for each of the window of interest and the number of samples related to the regression analysis processing are increased as well. According to the above configuration, it is possible to examine an optimum regression equation with a larger number of options. As a result, not only a regression equation that enables calculation of the estimated value Eb of the non-invasive blood pressure with higher accuracy can be selected, but also more detailed information as for the optimum window of interest and the optimum number of samples related to the regression analysis processing can be acquired.

The processor 143 of the estimating device 14 may be configured to keep the number of the regression analysis processings to be executed constant regardless of the increase of the number of measurements N, in a case where the number of the regression analysis processings to be executed reaches a prescribed value.

In the example illustrated in FIG. 10, the number of regression analysis processings to be executed when the 11th measurement of the non-invasive blood pressure is performed is the same as the number of regression analysis processings to be executed when the 10th measurement of the non-invasive blood pressure is performed. In other words, the processor 143 changes only the window of interest while maintaining the number of the regression analysis processings to be executed. Specifically, in the first regression analysis processing, 10 measurements included in the window of interest designating the 2nd to 11th measurements are sampled. In the second regression analysis processing, 9 measurements included in the window of interest designating the 3rd to 11th measurements are sampled. In the third regression analysis processing, 8 measurements included in the window of interest designating the 4th to 11th measurements are sampled. In the fourth regression analysis processing, 7 measurements included in the window of interest designating the 5th to 11th measurements are sampled.

Increasing the number of options of the regression equation to be examined can enhance the possibility of selecting an optimum regression equation, but accompanies an increase in the processing load of the processor 143. According to the above configuration, it is possible to suppress an increase in the processing load.

In other words, the value of the number of measurements N from which the regression analysis processing is performed plural times, and the value of the number of measurements N from which the number of the regression analysis processings to be executed is kept constant may be appropriately determined in accordance with expected calculation accuracy of the estimated value Eb of the non-invasive blood pressure, as well as the processing capability of the processor 143.

The processor 143 of the estimating device 14 may be configured to specify the number of samples (the number of measured values Mb of the non-invasive blood pressure) that are used in the regression analysis processing for acquiring the regression equation, as an optimum number of samples, upon selection of the optimum regression equation as described above. In this case, as illustrated in FIG. 1, the processor 143 is configured to output, from the output interface 144, sample number data SN for causing the information presenting device 13 of the monitoring device 10 to visibly present a relationship between the optimum sample number and the number of measurements N of the non-invasive blood pressure.

The information presenting device 13 is configured to visibly present the relationship between the optimum number of samples and the number of measurements based on the sample number data SN. FIG. 11 illustrates an example of the relationship between the optimum number of samples and the number of measurements, that is visibly presented in the information presenting device 13. FIG. 12 illustrates another example of the same relationship.

According to the above configuration, the hemodynamics of the subject 20 can be estimated based on the visibly-presented relationship between the optimum number of samples and the number of measurements. For example, as illustrated in FIG. 11, in a case where a tendency appears that the optimum sample number is decreased in accordance with an increase in the number of measurements, it may be estimated that an acute change occurs in the hemodynamics of the subject 20. From the tendency illustrated in FIG. 12, it may be estimated that no acute change occurs in the hemodynamics of the subject 20.

In a case where the regression analysis processing is performed with a plurality of explanatory variable parameters, the processor 143 of the estimating device 14 may be configured to acquire a statistic representing contribution from each of the explanatory variable parameters to the acquired regression equation. Examples of the statistic include an F value. In this case, as illustrated in FIG. 1, the processor 143 is configured to output, from the output interface 144, statistic data ST for causing the information presenting device 13 of the monitoring device 10 to visibly present the statistic.

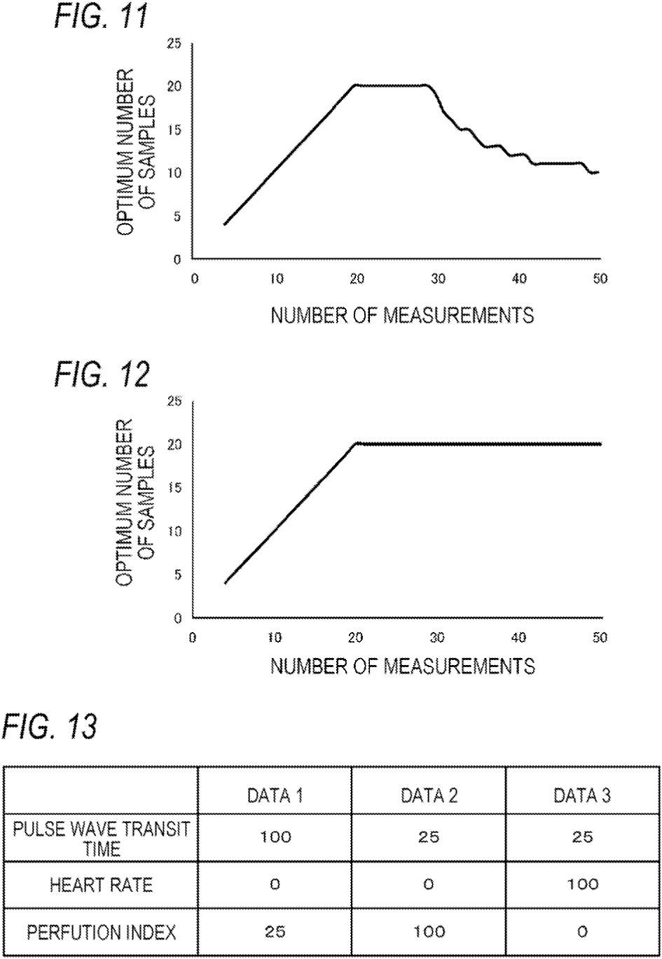

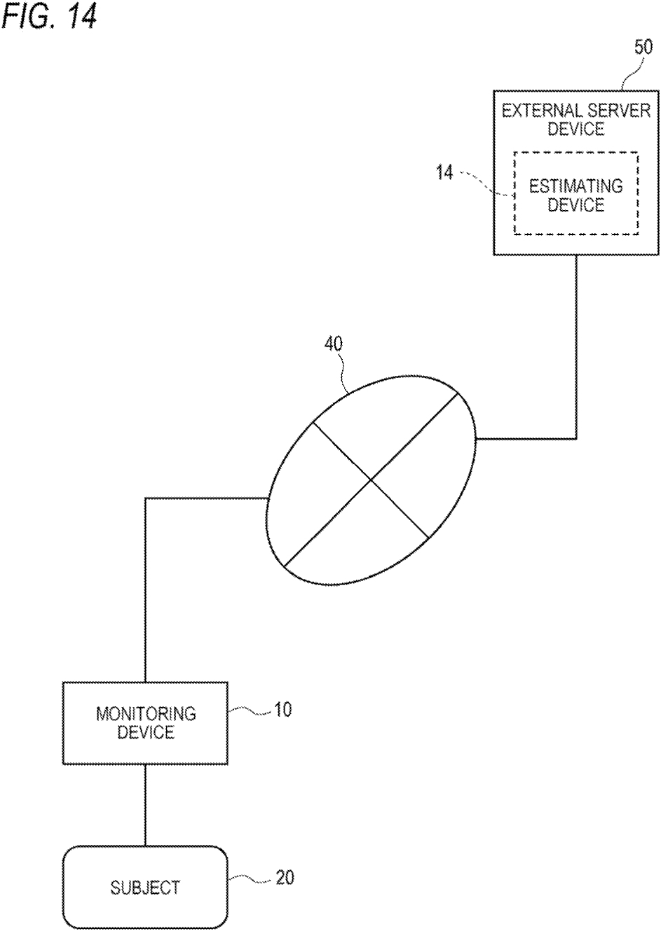

The information presenting device 13 is configured to visibly present the statistic based on the statistic data ST. FIG. 13 illustrates a summary of the statistics that are visibly presented by the information presenting device 13. The illustrated three data may be obtained by separate measurements performed on the same subject, or may be obtained by measurements performed on different subjects.

In this example, the summary of the statistics is visibly presented as a table indicating F values each of which has been normalized such that the value “50” represents a normal condition of the subject 20. However, the summary of the statistics may take the form of a bar graph, a pie graph, a radar chart, or the like.

According to the above configuration, mechanism of changes in the non-invasive blood pressure can be estimated based on the acquired statistic. For example, in a case where the contribution from the pulse wave transit time is high, mechanism that depends on the cardiac output is estimated. When the contribution from the heart rate is high, mechanism that depends on the stress is estimated. When the contribution from the perfusion index is high, mechanism that depends on the vascular resistance is estimated.

The processor 143 having various functions described above may be implemented by one or more general-purpose microprocessors configured to cooperate with one or more general-purpose memories. Examples of the general-purpose microprocessor include a CPU, an MPU, and a GPU. Examples of the general-purpose memory include a ROM and a RAM. In this case, a computer program for executing the above-described processing may be stored in the ROM. The ROM is an example of a non-transitory computer-readable medium having stored the computer program. The general-purpose microprocessor designates at least a part of the computer program stored in the ROM, loads the designated part in the RAM, and executes the above-described processing in cooperation with the RAM. The above-described computer program may be pre-installed in the general-purpose memory, or may be downloaded from an external server device 50 via a communication network 40 illustrated in FIG. 14 and installed in the general-purpose memory. In this case, the external server device 50 is an example of the non-transitory computer-readable medium having stored the computer program.

The processor 143 having various functions described above may be implemented by a dedicated integrated circuit capable of executing the above-described computer program, such as a microcontroller, an ASIC, and an FPGA. In this case, the above-described computer program is pre-installed in a memory element included in the dedicated integrated circuit. The memory element is an example of the non-transitory computer-readable medium having stored the computer program. The processor 143 may also be implemented by a combination of the general-purpose microprocessor and the dedicated integrated circuit.

The above embodiment is merely exemplary to facilitate understanding of the presently disclosed subject matter. The configuration according to the above embodiment can be appropriately modified or improved without departing from the gist of the presently disclosed subject matter.

In the above embodiment, the estimating device 14 is incorporated in the monitoring device 10. However, the estimating device 14 may be incorporated in the external server device 50 that can communicate with the monitoring device 10 via the communication network 40 illustrated in FIG. 14.

In this case, the first input interface 141 of the estimating device 14 is configured to include a communication interface that can receive the measured value Mb of the non-invasive blood pressure from the first measuring device 11 of the monitoring device 10 via the communication network 40. The second input interface 142 of the estimating device 14 is configured to include a communication interface that can receive the measured value Mp of the pulse wave transit time, the measured value Mh of the heart rate, and the measured value Mi of the perfusion index from the second measuring device 12 of the monitoring device 10 via the communication network 40. The output interface 144 of the estimating device 14 is configured to include a communication interface that can transmit various data to be used for visible presentation to the information presenting device 13 of the monitoring device 10 via the communication network 40.

In the above embodiment, a plurality of explanatory variable parameters are used in the regression analysis processing. However, the explanatory variable parameter to be used may be single.

In the above embodiment, the non-invasive blood pressure is selected as the objective variable of the regression analysis processing, and a plurality of explanatory variables are used. However, any physiological parameter for which intermittent measurements are performed may be selected as the objective variable. In addition, an adequate physiological parameter having a correlation with the physiological parameter selected as the objective variable may be selected as the explanatory variable. The photoplethysmogram sensor 32 and the electrocardiogram electrode 33 in the above embodiment may be appropriately changed in accordance with the selected physiological parameter.

The phrase “at least one of A and B” as used herein with respect to the two entities A and B is meant to include cases where only A is identified; only B is identified; and both A and B are identified. Each of the entities A and B may be singular or plural unless otherwise noted.

The phrase “at least one of A, B, and C” as used herein with respect to the three entities A, B, and C is meant to include cases where only A is identified; only B is identified; only C is identified; A and B are identified; B and C are identified; A and C are identified; and A, B, and C are all identified. Each of the entities A, B, and C may be singular or plural unless otherwise noted. The same applies to a case where the number of entities to be described is four or more.

The present application is based on Japanese Patent Application No. 2020-121471 filed on Jul. 15, 2020, the entire contents of which are hereby incorporated by reference.

1. An estimating device for a physiological parameter value, comprising:
a first interface configured to receive measured values of a first physiological parameter of a subject intermittently at a first frequency;
a second interface configured to receive measured values of n (n is an integer that is no less than 1) second physiological parameter of the subject at a second frequency higher than the first frequency; and
a processor configured to:
execute regression analysis processing based on the measured values of the second physiological parameter received by the second interface until when a measured value acquired by Nth (N is an integer that is no less than n+1) measurement of the first physiological parameter is received by the first interface in order to acquire a regression equation in which the measured value of the second physiological parameter is set as an explanatory variable;
calculate estimated values of the first physiological parameter with the regression equation and the measured values of the second physiological parameter received by the second interface, until a measured value acquired by (N+1)th measurement of the first physiological parameter is received by the first interface; and
output data for visibly presenting the estimated values.

2. The estimating device according to claim 1,
wherein the processor is configured to correct a constant term of the at least one regression equation such that the estimated value calculated when the measured value acquired by the Nth measurement of the first physiological parameter is received is made coincident with the measured value.

3. The estimating device according to claim 1,
wherein the n is no less than 2; and
wherein the processor is configured to:
execute the regression analysis processing with a plurality of regression equations;
select, from the regression equations, one regression equation used to calculate an estimated value that is closest to the measured value acquired by the Nth measurement of the first physiological parameter when the measured value is received; and
output data for visibly present the estimated value calculated with the one regression equation, until the measured value acquired by the (N+1)th measurement of the first physiological parameter is received.

4. The estimating device according to claim 1, wherein the processor is configured to:
acquire a plurality of regression equations by executing a plurality of regression analysis processings in which at least one of a window of interest designating which measurements is subjected to the regression analysis processing and number of measurements to be subjected to the regression analysis processing as samples is different from one another;
select, from the regression equations, one regression equation used to calculate an estimated value that is closest to the measured value acquired by the Nth measurement of the first physiological parameter when the measured value is received; and
output data for visibly present the estimated value calculated with the one regression equation, until the measured value acquired by the (N+1)th measurement of the first physiological parameter is received.

5. The estimating device according to claim 4,
wherein the processor is configured to increase number of the regression analysis processing to be executed in accordance with increase of the N.

6. The estimating device according to claim 5,
wherein the processor is configured to keep the number of the regression analysis processing to be executed irrespective of the increase of the N, in a case where the number of the regression analysis processing reaches a prescribed value.

7. The estimating device according to claim 4,
wherein the processor is configured to output data for visibly presenting relationship between the N and number of the measured values of the first physiological parameter that are used in the one regression equation.

8. The estimating device according to claim 1,
wherein the n is no less than 2; and
wherein the processor is configured to:
acquire statistics each of which represents contribution as the explanatory variant in the regression analysis processing from an associated one of n different second physiological parameters; and
output data for visibly presenting the statistics.

9. The estimating device according to claim 1,
wherein the first physiological parameter is non-invasive blood pressure; and
wherein the second physiological parameter includes at least one of pulse wave transit time, heart rate, and perfusion index.

10. A non-transitory computer-readable medium having stored a computer program adapted to be executed by a processor in an estimating device configured to estimate a physiological parameter value of a subject, the computer program being configured to cause, when executed, the estimating device to:
receive measured values of a first physiological parameter of a subject intermittently at a first frequency;
receive measured values of n (n is an integer that is no less than 1) second physiological parameter of the subject at a second frequency higher than the first frequency;
execute regression analysis processing based on the measured values of the second physiological parameter received until when a measured value acquired by Nth (N is an integer that is no less than n+1) measurement of the first physiological parameter is received in order to acquire a regression equation in which the measured value of the second physiological parameter is set as an explanatory variable;
calculate estimated values of the first physiological parameter with the regression equation and the measured values of the second physiological parameter, until a measured value acquired by (N+1)th measurement of the first physiological parameter is received by the first interface; and
output data for visibly presenting the estimated values.

11. A monitoring device, comprising:
a first interface configured to receive measured values of a first physiological parameter of a subject intermittently at a first frequency;
a second interface configured to receive measured values of n (n is an integer that is no less than 1) second physiological parameter of the subject at a second frequency higher than the first frequency;
a processor configured to execute regression analysis processing based on the measured values of the second physiological parameter received by the second interface until when a measured value acquired by Nth (N is an integer that is no less than n+1) measurement of the first physiological parameter is received by the first interface in order to acquire a regression equation in which the measured value of the second physiological parameter is set as an explanatory variable; and
an information presenting device configured to visibly present estimated values of the first physiological parameter calculated with the regression equation and the measured values of the second physiological parameter received by the second interface, until a measured value acquired by (N+1)th measurement of the first physiological parameter is received by the first interface.

12. An estimating method for a physiological parameter value, comprising:
receiving measured values of a first physiological parameter of a subject intermittently at a first frequency;
receiving measured values of n (n is an integer that is no less than 1) second physiological parameter of the subject at a second frequency higher than the first frequency;
executing regression analysis processing based on the measured values of the second physiological parameter received until when a measured value acquired by Nth (N is an integer that is no less than n+1) measurement of the first physiological parameter is received in order to acquire a regression equation in which the measured value of the second physiological parameter is set as an explanatory variable; and
presenting visibly estimated values of the first physiological parameter calculated with the regression equation and the measured values of the second physiological parameter, until a measured value acquired by (N+1)th measurement of the first physiological parameter is received by the first interface.

13. The monitoring device according to claim 11,
wherein the processor is configured to:
acquire a plurality of regression equations by executing a plurality of regression analysis processings in which at least one of a window of interest designating which measurements is subjected to the regression analysis processing and number of measurements to be subjected to the regression analysis processing as samples is different from one another;
select, from the regression equations, one regression equation used to calculate an estimated value that is closest to the measured value acquired by the Nth measurement of the first physiological parameter when the measured value is received; and
output data for visibly present the estimated value calculated with the one regression equation, until the measured value acquired by the (N+1)th measurement of the first physiological parameter is received.

14. The estimating device according to claim 13,
wherein the processor is configured to increase number of the regression analysis processing to be executed in accordance with increase of the N.

15. The estimating device according to claim 14,
wherein the processor is configured to keep the number of the regression analysis processing to be executed irrespective of the increase of the N, in a case where the number of the regression analysis processing reaches a prescribed value.
